(19) United States
(12) Patent Application Publication
(10) Pub. No.: US 2004/0034243 A1
(43) Pub. Date: Feb. 19, 2004

(54) Method for producing poly-or monomethylol alkanoic acids

(76) Inventors: Hagen Weigl, Ladenburg (DE); Klaus Ebel, Lampertheim (DE); Steffen Maas, Bubenheim (DE); Gerhard Schulz, Bad Durkheim (DE); Matthias Dernbach, Dossenheim (DE)

Correspondence Address: KEIL & WEINKAUF, 1350 CONNECTICUT AVENUE, N.W., WASHINGTON, DC, 20036, US

(21) Appl. No.: 10/399,493

(22) PCT Filed: Oct. 31, 2001
(86) PCT No.: PCT/EP01/12563

(30) Foreign Application Priority Data
Nov. 8, 2000 (DE) 10055181.5

(51) Int. Cl. C07C 51/16; C07C 51/34; C07C 51/235

(52) U.S. Cl. 554/132; 562/531

(57) ABSTRACT

Polymethylolalkanoic or monomethylolalkanoic acids of the formula (I)

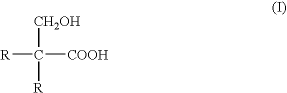

where R may be identical or different and are each a substituted or unsubstituted aliphatic hydrocarbon group having from 1 to 22 carbon atoms, an aryl or arylalkyl group having from 6 to 22 carbon atoms or a methylol group, are prepared from the corresponding polymethylolalkanals or monomethylolalkanals of the formula (II)

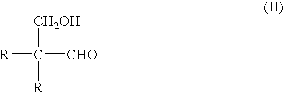

where R is as defined above, by oxidation using hydrogen peroxide. The polymethylolalkanal or monomethylolalkanal of the formula (II) used in the reaction has a total content of metal ions of groups 3 to 14 of the Periodic Table of the Elements of up to 5 ppm.

The process is particularly useful for preparing dimethylolalkanoic acids from the corresponding dimethylolalkanals, especially for preparing dimethylolbutanoic or dimethylolpropanoic acid.

The present invention relates to a process for preparing polymethylolalkanoic or monomethylolalkanoic acids, in particular dimethylolalkanoic acids.

It is known that polymethylolalkanoic acids can be prepared from the corresponding polymethylolalkanals by oxidation using hydrogen peroxide. U.S. Pat. No. 3,312,736 uses a molar ratio of hydrogen peroxide to polymethylolalkanal of from about 0.4 to 1 for this purpose. The pH is kept in the range from 3 to about 9 after addition of the hydrogen peroxide and the reaction temperature is held at a maximum of 95° C.

The preparation of dimethylolpropionic acid by oxidation of dimethylolpropanal by means of H₂O₂ is known via Chemical Abstract 131: 20530 from Pige Huagong (1998), 15 (6), 27-29, a technical report from the Institute for Applied Chemistry of the University of Anhui in Hefei, People's Republic of China. Here, H₂O₂ is added at from 50 to 60° C. to dimethylolpropanal and the resulting reaction mixture is heated stepwise to 95° C.

A further technical report is known via Chemical Abstract 130: 326533 from Jingxi Huagong (1999), 16 (1), 28-31, likewise from the Institute for Applied Chemistry of Anhui University, which states a temperature of 95° C. and a molar ratio of H₂O₂ to propionaldehyde of 1.1:1 as optimum reaction conditions for the oxidation. The propionaldehyde is initially converted into dimethylolpropanal in an aldol reaction with formaldehyde using NaOH as basic catalyst.

All the abovementioned processes have the disadvantage that the selectivity is unsatisfactory, which leads to a relatively high by-product content and thus to a reduced yield of the desired acid.

It is an object of the present invention to provide a process for preparing polymethylolalkanoic acids or monomethylolalkanoic acids by means of which the selectivity can be increased significantly so as to reduce the by-product content and to increase the yield of desired polymethylolalkanoic acid or monomethylolalkanoic acid.

We have found that this object is achieved by a process for preparing polymethylolalkanoic or monomethylolalkanoic acids of the formula (I)

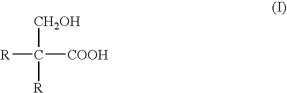

where R may be identical or different and are each a substituted or unsubstituted aliphatic hydrocarbon group having from 1 to 22 carbon atoms, an aryl or arylalkyl group having from 6 to 22 carbon atoms or a methylol group, from the corresponding polymethylolalkanals or monomethylolalkanals of the formula (II)

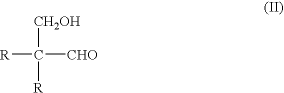

where R is as defined above, by oxidation using hydrogen peroxide, wherein the polymethylolalkanal or monomethylolalkanal of the formula (II) has a total content of metal ions of groups 3 to 14 of the Periodic Table of the Elements of up to 5 ppm.

The polymethylolalkanal or monomethylolalkanal of the formula (II) preferably comprises iron(II), iron(III), chromium(III), chromium(IV) and nickel(II) as elements of groups 3 to 14 of the Periodic Table of the Elements. The total content of such metal ions in the polymethylolalkanals or monomethylolalkanals of the formula (II) should not exceed 5 ppm, with the content of each metal ion being, depending on the number of metal ions present, from 0.001 to 5 ppm, preferably from 0.001 to 2 ppm.

The process for preparing the polymethylolalkanoic acids can be carried out by reacting the polymethylolalkanals required as starting compounds either as pure substances or in admixture with other compounds. Since the corresponding polymethylolalkanals are prepared, for example, by aldol reaction of the corresponding aliphatic aldehydes with formaldehyde in the presence of a basic catalyst, the crude reaction product from this reaction could be passed directly to the oxidation reaction to form the polymethylolalkanoic acid.

A prepurification of this crude reaction product is known, for example, from the German patent application No. 199 63 445.9, in which the removal of formaldehyde is described. In this context, reference is made, in particular, to Examples 2-4 in so far as they concern the distillation of the aldolization product, i.e. of the polymethylolalkanal of the formula (II). The reaction conditions mentioned in the examples are applicable in an analogous manner to monomethylolalkanals. Removal of metal ions from the crude reaction product is not described. In addition, polymethylolalkanal or monomethylolalkanal is lost as a result of the distillation.

According to the present invention, it is now recognized that the yield in the oxidation of the polymethylolalkanals or monomethylolalkanals can be improved considerably if the metal ion content of the polymethylolalkanal or monomethylolalkanal is limited without polymethylolalkanal or monomethylolalkanal being lost in the process.

Metal ions generally get into the polymethylolalkanal or monomethylolalkanal as a result of corrosion of plant components in the aldolization. Polymethylolalkanal or monomethylolalkanal having a reduced metal ion content can therefore be obtained, as one possibility, by avoiding introduction of metal ions, for example by choice of suitable metal-free materials such as glass or enamel, or high-performance materials such as titanium or high-performance alloys, for storage and intermediate vessels, pipes, reactors, distillation columns and rectification columns. Since this can, if it is technically feasible at all, be associated with considerable costs, especially for industrial plants, preference is given to reducing the metal ion content by removal of metal ions from the polymethylolalkanals or monomethylolalkanals and/or from the starting materials required for their preparation or the process streams in which they are present, for example in the aldol reaction for preparing the polymethylolalkanals or monomethylolalkanals from the corresponding aliphatic aldehydes and formaldehyde.

The removal of metal ions from the polymethylolalkanals or monomethylolalkanals and/or from the starting materials such as formaldehyde or aliphatic aldehydes required for their preparation can be achieved by treatment with absorbents and/or by complexation and subsequent membrane separation processes, preferably by treatment with absorbents.

In the complexation with subsequent membrane separation processes according to the present invention, a highly polymeric, soluble complexing agent which complexes the metal ions present in the feed is added. As complexing agents, it is possible to use polymers of any type which contain functional groups capable of complexation (for example COOH, NR₂, etc.) or heteroatoms such as N or P. Thus, for example, it is possible to use polyimines of appropriate molar mass. The complexed polymer and the excess of uncomplexed polymer is subsequently removed from the hydrogenation feed by means of a suitable membrane (organic or inorganic). This membrane holds back the complexing agent together with bound metal ions while the polymethylolalkanal or monomethylolalkanal passes through the membrane and is subsequently oxidized.

As absorbents, preference is given to using activated carbon, acid or base ion exchangers or mixtures thereof, metal oxides or molecular sieves, with particular preference being given to chelating ion exchangers.

According to the present invention, it has been observed that no decomposition of the sensitive polymethylolalkanals occurs on treatment with the absorbents. This is surprising since it is known that polymethylolalkanals tend to undergo a retro-aldol reaction, i.e. the reverse reaction to their formation, at elevated temperature and when treated with acids or bases.

Suitable activated carbons have, for example, a surface area of from 500 to 2000 m²/g measured in accordance with DIN 66 131 and a porosity of from 0.05 to 1.0 cm³/g measured in accordance with DIN 66134 and are marketed by Merck, Darmstadt, by Chemviron Midwest Corp., Wooster, USA, under the trade name CPG LF 8 30® and by Lurgi AG, Frankfurt, under the trade name Carboraffin P®.

Suitable ion exchangers are, for example, the strong acid ion exchanger IR® 120 marketed by Merck, Darmstadt, preferably chelating ion exchangers such as Amberlite® TRL from Rohm & Haas, Darmstadt, Levatit® TP 207 from Bayer AG, Leverkusen, Chelese® 100 from Merck, Darmstadt, in all possible forms, for example particulate or as gel.

Metal oxides which can be used are, for example, alpha- or gamma-aluminum oxide, silicon oxide, titanium dioxide in the anatase or rutile modification, zirconium dioxide, magnesium oxide, calcium oxide, zinc oxide or mixtures of metal oxides such as aluminosilicates. A suitable aluminum oxide is marketed, for example, by Condea Chemie AG, Hamburg, under the trade name Pural®SB.

Suitable molecular sieves are, for example, aluminosilicates or zeolites having a pore diameter of greater than 3 A, for example Zeokat ®Z 6 01-01-y zeolite or Zeochem® molecular sieve 13×13 zeolite from Uetikon AG, Switzerland.

The absorbents can be used in the form of shaped bodies such as spheres, extrudates, pellets, granules or powder.

In general, the polymethylolalkanal or monomethylolalkanal and/or the starting material necessary for its preparation is/are treated at a temperature between the solidification point and the boiling point of the polymethylolalkanal or monomethylolalkanal or the starting material required for its preparation, whose metal ion content is to be reduced, preferably at from 20 to 150° C., particularly preferably from 40 to 100° C., and pressures of from 0.001 to 200 bar, preferably from 0.5 to 10 bar, in a stirred vessel, but preferably by passage through an absorbent in a fixed bed.

In a particularly preferred embodiment, the polymethylolalkanals or monomethylolalkanals are prepared from the corresponding aliphatic aldehydes and formaldehyde by an aldol reaction in the presence of a basic catalyst as described in WO 98/28253, which is hereby expressly incorporated by reference. The crude reaction product from the aldolization as described in WO-98/28253 is, preferably on the side still at atmospheric pressure, passed directly over an absorbent in a fixed bed, particularly preferably a chelating ion exchanger, and then passed to the oxidation to form the corresponding dimethylolalkanoic acids. When using ion exchangers as absorbent, the manufacturer's recommendations for the optimum temperature range of the respective ion exchanger should be observed within the abovementioned temperature ranges.

The residence time of the polymethylolalkanal or monomethylolalkanal or the starting material necessary for its preparation is dependent on the affinity of the absorbent and is generally in the range from 1 minute to 24 hours, preferably from 5 to 30 minutes. It is possible to introduce the polymethylolalkanal or monomethylolalkanal or the starting material required for its preparation in a solvent, preferably in the form of a 20-60% strength by weight solution. Suitable solvents are, for example, water, alcohols such as methanol and ethanol, or 0.1-99% strength alcohol/water mixtures. The treatment with the absorbent is preferably carried out in water or a 0.1-99% strength alcohol/water mixture.

The absorbent can be regenerated, for example by flushing it depending on the absorbent, with water, alkalis or acids and subsequently washing it with water. Acid or strong acid ion exchangers are preferably regenerated using aqueous hydrochloric acid, sulfuric acid, formic acid or acetic acid, while base or strong base ion exchangers are preferably regenerated using aqueous sodium hydroxide, potassium hydroxide or Ca(OH)₂.

The avoidance of the introduction of metal ions is particularly preferably combined with the treatment of the polymethylolalkanals or monomethylolalkanals or the starting materials required for their preparation with an absorbent.

In a preferred embodiment of the invention, a distinction is made between an initial phase and a final phase in the oxidation, where the reaction temperature during the initial phase is chosen so as to be lower than during the final phase and is at least=40° C. and the temperature during the final phase is at least >85° C.

In a batch or semibatch reaction in which the polymethylolalkanal or monomethylolalkanal to be oxidized is placed in a reaction vessel and hydrogen peroxide is metered in, the initial phase of the reaction is at least the reaction time which is necessary for adding the hydrogen peroxide to the alkanal. In a continuous reaction, the initial phase is defined as at least the time in which the components are metered in, with the initial phase being at least around 1% of the reaction or residence time of the reaction mixture.

The final phase for the purposes of the present invention is the reaction or residence time during which the reaction components are present in a mixed state in the desired ratio and are allowed to react further at a temperature higher than in the initial phase, i.e. at a temperature above 85° C.

As a result of the stepped temperatures which are preferably selected, the reaction proceeds more selectively than under the reaction conditions known from the prior art. In addition, a reaction temperature of at least 40° C. and preferably 60° C. during the initial phase results in the reaction with hydrogen peroxide proceeding more rapidly and counters accumulation, so that the reaction conditions according to the present invention also bring safety advantages. Furthermore, evaporative cooling can be achieved at this initial temperature, even under slightly subatmospheric pressure, which can be exploited advantageously for carrying out the reaction. Thus, the enthalpy of vaporization of the solvent can be utilized to remove the large quantity of heat of reaction so that more rapid introduction is possible and the reaction can be carried out independently of other heat exchangers. Furthermore, solvent can be removed in this way without additional input of energy. However, the reaction temperature during the initial phase must not be too high, since hydrogen peroxide is decomposed at temperatures of >95° C. without bringing about the desired oxidation reaction.

On the other hand, relatively high reaction temperatures during the final phase of the oxidation reaction make it possible to achieve shorter reaction times. Furthermore, intermediate compounds which are formed during the reaction can react, since the necessary activation energy is available. This in turn gives a significantly higher selectivity in the reaction.

The temperature during the initial phase is preferably kept within a range from 60 to 80° C., particularly preferably from 65 to 75° C. During the final phase, the reaction temperature is from >85 to 110° C., preferably from >85 to 105° C. The change of temperature between the initial phase and the final phase can take place as quickly as possible.

Although the process is suitable in principle for the preparation of polymethylolalkanoic or monomethylolalkanoic acids, it is preferably employed for the preparation of dimethylolalkanoic acids from the corresponding dimethylolalkanals. Dimethylolalkanoic acids and dimethylolalkanals in which the aliphatic hydrocarbon radical R has from 1 to 5 carbon atoms are of particular importance. Particular preference is given to using dimethylolalkanals whose aliphatic radical has one or two carbon atoms, i.e. dimethylolpropanal or dimethylolbutanal, as starting compounds to be oxidized.

The oxidant used is an aqueous solution of hydrogen peroxide having a hydrogen peroxide content of from 5 to 60%, preferably from 30 to 50%. This concentration range advantageously provides a minimum amount of oxidant per unit volume of solvent. It has been found that too much water present at the end of the reaction greatly hinders later work-up due to the high solubility of the desired end product, and may even require a further work-up step, e.g. concentration by evaporation. On the other hand, it has been found that very high concentrations of hydrogen peroxide, i.e. concentrations above 60%, offer little advantage because the hydrogen peroxide then tends to decompose.

With regard to the stoichiometric ratios, i.e. the ratio of hydrogen peroxide added as oxidant to the compound to be oxidized, it is known from the prior art that either the amount of dimethylolalkanal or the amount of aliphatic aldehyde which is subsequently reacted in an aldol reaction to form the dimethylolalkanal can be used as starting point for the stoichiometric calculations. It is also known that adherence to a defined stoichiometric ratio has significant effects on the yield. In the context of the present invention, it has now been found that the significantly higher selectivity achieved in the reaction carried out according to the present invention is also due to the different method of calculating the stoichiometric ratios compared to the prior art. According to the invention, the amount of formaldehyde and dimethylolpropionaldehyde in the reaction solution at the beginning of the oxidation reaction is always employed as a basis for the calculation of the stoichiometric ratio.

The ratio of hydrogen peroxide used to the sum of these aldehyde compounds is, according to the present invention, from 0.5 to 0.99, preferably from 0.7 to 0.99, particularly preferably from 0.85 to 0.95: i.e. the oxidant is used in a substoichiometric amount based on the amount of the compounds to be oxidized.

The polymethylolalkanoic acids can be prepared in a purity of >96% by means of the process of the present invention. These are subsequently isolated in the stated purity by crystallization from the reaction mixture, solid/liquid separation and subsequent washing with water or another suitable solvent or solvent mixture without further purification steps, e.g. extraction, passage through an ion exchanger or the like, being necessary. This makes the process of the present invention less complicated. Buffering of the reaction solution is also unnecessary in the present process.

The filtrate obtained from the solid/liquid separation can be concentrated further by distillation at atmospheric pressure or under reduced pressure, so that more of the polymethylolalkanoic acid concerned is obtained in crystalline form on cooling. This can be separated from the mother liquor by solid/liquid separation. The mother liquor obtained in this way can in turn be concentrated and processed further. The purity of the crystalline product can be increased by washing or recrystallization. However, it is also possible to omit purification and to recycle the crystallized material to the next reaction pass upstream of the crystallization. This achieves a purification effect without an additional process stage having to be provided.

Likewise, the washing water from the first crystals, which can still contain end product because of the sometimes high solubility of the polymethylolalkanoic acids, can be recycled to the next pass. The solvent which is thus recirculated simultaneously with the end product can then be removed by distillation at atmospheric pressure or under reduced pressure.

The mother liquor obtained by work-up, one or more times, has a significantly reduced water content. Disposal by means of incineration is thus considerably less expensive, since less water has to be heated and vaporized.

The invention will now be illustrated by means of examples.

### EXAMPLES

### Example 1

Preparation of Dimethylolpropionaldehyde

540 g of aqueous formaldehyde (30% strength, 5.4 mol) and 19.7 g of 45% strength aqueous trimethylamine are placed in a reaction vessel and 174.0 g of propionaldehyde (3.0 mol) are metered in over a period of 30 minutes, with the temperature being held in the range from 40° C. to 45° C. by cooling. The mixture is then stirred for another 1 hour at 40° C. and subsequently for 2 hours at 70° C. 67 g of distillate are taken off from the reaction mixture at atmospheric pressure. This leaves, as bottoms, an aqueous solution comprising 37.2% of dimethylolpropionaldehyde and 2% of formaldehyde, corresponding to a dimethylolpropionaldehyde yield of 77.8% based on formaldehyde.

Determination of the Metal Ion Content

A 0.5 to 1 g sample of a polymethylolalkanal or monomethylolalkanal was subjected to acid digestion in which the sample is admixed and then heated to boiling firstly with 0.2 ml of an aqueous Na₂SO₄ solution (200 g/l of Na₂SO₄), then with 8 ml of sulfuric acid (1.84 g/ml) and subsequently with 3 ml of nitric acid (1.41 g/ml). The digested sample is then oxidized hot with 10 ml of a mixture of nitric acid, sulfuric acid and perchloric acid in a volume ratio of 2:1:1. After fuming off the excess acids, the residue is made up to 10 ml with dilute hydrochloric acid. The metal ion concentration is determined in this solution by inductively coupled plasma atomic emission spectroscopy (ICP-AES), for example using the IRIS Advantage ICP spectrometer from Thermo-Jarrel Ash.

### Examples 2 to 4 and Comparative Examples C1 to C3

In each case, 80 g of an aqueous solution of dimethylolpropionaldehyde (37.2% by weight; 0.25 mol) containing iron ions in the amounts indicated in Table 1 were admixed with 18.8 g of hydrogen peroxide solution (50% strength by weight) at from 67 to 71° C. and a pressure of 400 mbar. The hydrogen peroxide solution was metered in over a period of 30 minutes. The pressure was then increased to atmospheric pressure and the mixture was heated to 100° C. This temperature was maintained for 3 hours and the dimethylolpropionic acid content was subsequently determined by gas chromatography. The yields of dimethylolpropionic acid obtained are shown in Table 1 below.

TABLE 1

| | Concentration of Fe | |
|---|---|---|
| Example No. | [ppm] | Yield [%] |
| 2 | 0.001 | 79.1 |
| 3 | 2 | 73.9 |
| 4 | 5 | 72.6 |
| C1 | 7 | 71.7 |
| C2 | 10 | 70.5 |
| C3 | 25 | 67.1 |

Comparison of the Examples 1 to 3 according to the present invention with the Comparative Examples C1 to C3 clearly shows the effect of the metal ion content on the reaction of dimethylolpropanal with hydrogen peroxide.

### Examples 4 and 5, Comparative Example C4

A 40.6% strength by weight aqueous dimethylolpropionaldehyde solution prepared as described in Example 1 and in each case containing 21 ppm of iron ions was stirred at room temperature with 10% by weight of molecular sieves of differing pore sizes. After 15 hours, the iron ion content was checked by analysis. The result is summarized in Table 2.

TABLE 2

| Example | Pore size (Å) | Fe [ppm] |
|---|---|---|
| Starting material | | 21 |
| 4 | 10 | 4 |
| 5 | 5 | 5 |
| C4 | 3 | 15 |

### Examples 5 to 14

A 40.6% strength by weight aqueous dimethylolpropionaldehyde solution prepared as described above and in each case containing 21 ppm of iron ions was stirred with various ion exchangers at room temperature. After 15 hours, the iron ion content was checked by analysis. The result is summarized in Table 3.

TABLE 3

| Exam- | | | | | |
|---|---|---|---|---|---|
| ple | Ion exchanger | Property | Time | | Fe [ppm] |
| 5 | Chelese ® 100 | weak acid | 16 | h | 1 |
| 6 | Chelese ® 100 | weak acid | 15 | min | 2 |
| 7 | Chelese ® 100 | weak acid | 30 | min | <1 |
| 8 | IR 120 | strong acid | 16 | h | 0.5 |
| 9 | IR 120 | strong acid | 2 | h | 2 |
| 10 | IR 120 | strong acid | 3 | h | 1 |
| 11 | IR 120 | strong acid | 5 | h | <1 |
| 12 | Chelating ion | | 4 | h | <1 |
| | exchanger (Lewatit ® | | | | |
| | TP 207) | | | | |
| 13 | Chelating ion | | 4 | h | <1 |
| | exchanger | | | | |
| | (Amberlite ® IRA 402) | | | | |
| 14 | Mixed-bed ion | mixed bed | 16 | h | 1 |
| | exchanger | | | | |
| | (Amberlite ® MB3) | | | | |

### Examples 15 and 16

A 40.6% strength by weight aqueous dimethylolpropionaldehyde solution prepared as described above and in each case containing 20 ppm of iron ions was stirred with 10% by weight of metal oxides at room temperature. After 18 hours, the iron ion content was checked by analysis. The result is summarized in Table 4.

TABLE 4

| Example | Absorbent | Fe [ppm] |
|---|---|---|
| 15 | γ-Al₂O₃ | 5 |
| 16 | Pural ® SB | 3 |

### Examples 17 to 18

A 40.6% strength by weight aqueous dimethylolpropionaldehyde solution prepared as described above and in each case containing 21 ppm of iron ions was stirred with 10% by weight of activated carbon at room temperature. After 15 hours, the iron ion content was checked by analysis. The result is summarized in Table 5.

TABLE 5

| Example | Absorbent | Fe [ppm] |
|---|---|---|
| 16 | Activated carbon powder | 1 |
| | (Merck) | |
| 17 | Carboparraffin ® P (Lurgi) | 0.5 |

1. A process for preparing polymethylolalkanoic or monomethylolalkanoic acids of the formula (I) 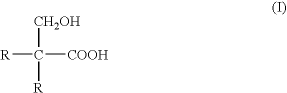
where R may be identical or different and are each a substituted or unsubstituted aliphatic hydrocarbon group having from 1 to 22 carbon atoms, an aryl or arylalkyl group having from 6 to 22 carbon atoms or a methylol group, from the corresponding polymethylolalkanals or monomethylolalkanals of the formula (II) 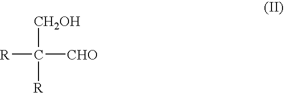
where R is as defined above, by oxidation using hydrogen peroxide, wherein the polymethylolalkanal or monomethylolalkanal of the formula (II) has a total content of metal ions of groups 3 to 14 of the Periodic Table of the Elements of up to 5 ppm.

2. A process as claimed in claim 1, wherein the content of each metal ion of groups 3 to 14 of the Periodic Table of the Elements is from 0.001 to 5 ppm.

3. A process as claimed in claims 1 or 2, wherein the metal ion content of the polymethylolalkanal or monomethylolalkanal is set by removing the metal ions from the polymethylolalkanals or monomethylolalkanals and/or the starting materials required for their preparation by treatment with absorbents and/or by complexation and subsequent membrane separation processes and/or avoidance of the introduction of the metal ions.

4. A process as claimed in any of claims 1 to 3, wherein the absorbent is selected from among activated carbon, acid or base ion exchangers and mixtures thereof, metal oxides and molecular sieves.

5. A process as claimed in claim 4, wherein the absorbent is a chelating ion exchanger.

6. A process as claimed in any of claims 1 to 5, wherein a distinction is made between an initial phase and a final phase in the reaction, where the reaction temperature during the initial phase is selected so as to be lower than during the final phase and is at least ≧40° C. and the temperature during the final phase is at least >85° C.

7. A process as claimed in any of claims 1 to 6 for preparing dimethylolalkanoic acids from the corresponding dimethylolalkanals.

8. A process as claimed in any of claims 1 to 7, wherein the aliphatic hydrocarbon group has from one to five carbon atoms.

9. A process as claimed in any of claims 1 to 8, wherein the aliphatic hydrocarbon group has one or two carbon atoms.

10. A process as claimed in any of claims 1 to 9, wherein the oxidation is carried out using an aqueous solution of hydrogen peroxide having a hydrogen peroxide content of from 5 to 60% by weight, preferably from 30 to 50% by weight.

11. A process as claimed in any of claims 1 to 10, wherein hydrogen peroxide is used in a substoichiometric amount, based on the molar ratio of hydrogen peroxide to the amount of formaldehyde and polymethylolalkanal present in the reaction solution at the beginning.

12. A process as claimed in any of claims 1 to 11, wherein the molar ratio is from 0.5 to 0.99, preferably from 0.7 to 0.99, particularly preferably from 0.85 to 0.95.

13. A process as claimed in claim 1, wherein the temperature during the initial phase is from >40 to 85° C., preferably from 60 to 80° C., particularly preferably 65-75° C., and during the final phase is from >85 to 110° C., preferably up to 105° C.
